(19) United States
(12) Patent Application Publication
(10) Pub. No.: US 2014/0309518 A1
(43) Pub. Date: Oct. 16, 2014

(54) MEDICAL IMAGING APPARATUS, CONTROL METHOD THEREOF, AND IMAGE PROCESSING APPARATUS FOR THE SAME

(71) Applicants: SAMSUNG ELECTRONICS CO., LTD., Suwon-si (KR); SAMSUNG LIFE PUBLIC WELFARE FOUNDATION, Seoul (KR)

(72) Inventors: Young Hun SUNG, Hwaseong-si (KR); Jae Hak LEE, Yongin-si (KR); Ho Young LEE, Suwon-si (KR); Myung Jin CHUNG, Seoul (KR)

(73) Assignees: SAMSUNG LIFE PUBLIC WELFARE FOUNDATION, Seoul (KR); SAMSUNG ELECTRONICS CO., LTD., Suwon-si (KR)

(21) Appl. No.: 14/250,584

(22) Filed: Apr. 11, 2014

(30) Foreign Application Priority Data
Apr. 11, 2013 (KR) 10-2013-0040031
Oct. 4, 2013 (KR) 10-2013-0118316

(51) Int. Cl. A61B 6/00; A61B 5/00; A61B 5/055

(52) U.S. Cl. CPC A61B 6/5211; A61B 6/48; A61B 5/055; A61B 5/7235
USPC 600/410; 600/407; 600/425

(57) ABSTRACT

Provided is a medical imaging apparatus including a scanner configured to acquire projection data of an object, a three dimensional (3D) recovery module configured to recover a volume of the object based on the projection data, a two dimensional (2D) image generator configured to generate a 2D image of the object based on the volume of the object, a 3D image generator configure to generate a 3D image of the object based on the volume of the object, a 2D display configured to display the 2D image of the object, and a 3D display configured to display the 3D image of the object.

### CROSS-REFERENCE TO RELATED APPLICATION

This application claims priority Korean Patent Application Nos. 2013-0040031 and 2013-0118316, filed on Apr. 11, 2013, and Oct. 4, 2013, respectively, in the Korean Intellectual Property Office, the disclosure of which is incorporated herein by reference.

### BACKGROUND

1. Field

Apparatuses and methods consistent with exemplary embodiments relate to a medical imaging apparatus which images the inside of an object two dimensionally and three dimensionally, a control method thereof, and an image processing apparatus for the same.

2. Description of the Related Art

Medical imaging apparatuses, such as a computed tomography (CT) apparatus, a positron emission tomography (PET) apparatus, a tomosynthesis apparatus, and a magnetic resonance imaging (MRI) apparatus, radiate radioactive rays or apply a magnetic field to an object to image the inside of the object non-invasively.

Particularly, the medical imaging apparatus may generate a three dimensional (3D) volume data together with two dimensional (2D) sectional images of an object. The 3D volume data allow a user to identify morphological characteristics of the inside of the object, and may thus be used in a diagnosis field.

However, since the 3D volume data may be seen as a 2D image from a certain viewpoint through volume rendering or seen as 2D images of slices, it may be difficult to identify an overall structure of the object at a glance and a degree of overlap between materials of the object in a depth direction.

### SUMMARY

One or more exemplary embodiments provide a medical imaging apparatus which includes a two dimensional (2D) display device configured to display a 2D image of an object and a three dimensional (3D) display device configured to display a 3D image of the object such that the 2D image and the 3D image may be identified during diagnosis to promote accuracy and promptness of diagnosis, a control method thereof, and an image processing apparatus for the same.

According to an aspect of an exemplary embodiment, a medical imaging apparatus includes a scanner configured to acquire projection data of an object, a 3D recovery module configured to recover a volume of the object based on the projection data, a 2D image generator configured to generate a 2D image of the object based on the volume of the object, a 3D image generator configured to generate a 3D image of the object based on the volume of the object, a 2D display configured to display the 2D image of the object, and a 3D display configured to display the 3D image of the object.

The scanner may acquire a plurality of projection data from a plurality of different viewpoints.

The 3D recovery module may include a tomographic image generator configured to generate a plurality of tomographic images of the object by reconstructing the projection data and a volume data generator configured to generate a volume data corresponding to the volume of the object based on the plurality of tomographic images.

The 2D image generator may generate at least one reprojection image corresponding to at least one viewpoint by rendering the volume of the object from the at least one viewpoint.

The 2D image generator may generate at least one sectional image corresponding to at least one plane from the volume of the object.

The 3D image generator may generate a plurality of reprojection images corresponding to a plurality of different viewpoints by rendering the volume of the object from the plurality of viewpoints.

The 3D image generator may generate a multi-view 3D image based on the plurality of reprojection images.

The plurality of reprojection images may include a first reprojection image corresponding to a left viewpoint and a second reprojection image corresponding to a right viewpoint.

The 3D display may substantially simultaneously display the first reprojection image corresponding to the left viewpoint and the second reprojection image corresponding to the right viewpoint

The 3D display may alternately display the first reprojection image corresponding to the left viewpoint and the second reprojection image corresponding to the right viewpoint

According to an aspect of another exemplary embodiment, a control method of a medical imaging apparatus includes acquiring projection data of an object, recovering a volume of the object based on the projection data, generating a 2D image of the object based on the volume of the object, generating a 3D image of the object based on the volume of the object, and displaying the 2D image of the object through a 2D display, and displaying the 3D image of the object through a 3D display.

According to an aspect of another exemplary embodiment, an image processing apparatus includes at least one processor which implements: a three dimensional (3D) recovery module configured to obtain a volume of an object based on a plurality of tomographic images of the object scanned from a plurality of viewpoints; an image generator configured to generate at least one from among a two dimensional (2D) image and a three dimensional (3D) image of the object based on the volume of the object.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a control block diagram of a medical imaging apparatus according to an exemplary embodiment;

FIG. 2A is a perspective view illustrating an external appearance of a medical imaging apparatus including a scanner which performs computed tomography (CT) according to an exemplary embodiment;

FIG. 2B is a sectional view of a radiation source which radiates X-rays according to an exemplary embodiment;

FIGS. 3A and 3B are views illustrating an external appearance of a medical imaging apparatus including a scanner which performs tomosynthesis according to exemplary embodiments;

FIG. 3C is a view illustrating a structure of a radiation detector which detects X-rays according to an exemplary embodiment;

FIG. 4 is a view illustrating an external appearance of a medical imaging apparatus including a scanner which performs magnetic resonance imaging;

FIG. 5 is a control block diagram illustrating a three dimensional (3D) recovery module according to an exemplary embodiment;

FIG. 6A is a schematic view illustrating tomographic images of an object according to an exemplary embodiment;

FIG. 6B is a schematic view illustrating a recovered volume of the object according to an exemplary embodiment;

FIG. 7 is a schematic view illustrating a volume of an object rendered from a viewpoint according to an exemplary embodiment;

FIGS. 8A and 8B are schematic views illustrating a sectional image generated from a volume of an object according to various exemplary embodiments;

FIG. 9 is a schematic view illustrating a process of rendering a volume from a right viewpoint and a left viewpoint according to an exemplary embodiment;

FIG. 10 is a control block diagram illustrating a configuration of a 3D display according to an exemplary embodiment;

FIG. 11 is a control block diagram illustrating a configuration of a 3D image generator when a multi-view method is employed according to an exemplary embodiment;

FIG. 12 is a schematic view illustrating a process of generating a plurality of reprojection images by rendering a volume of an object according to an exemplary embodiment;

FIG. 13 is a schematic view illustrating a process of combining a plurality of reprojection images to generate a multi-view 3D image according to an exemplary embodiment; and

FIG. 14 is a flowchart illustrating a control method of a medical imaging apparatus according to exemplary embodiment.

### DETAILED DESCRIPTION

Reference will now be made in detail to certain exemplary embodiments, which are illustrated in the accompanying drawings, wherein like reference numerals refer to like elements throughout.

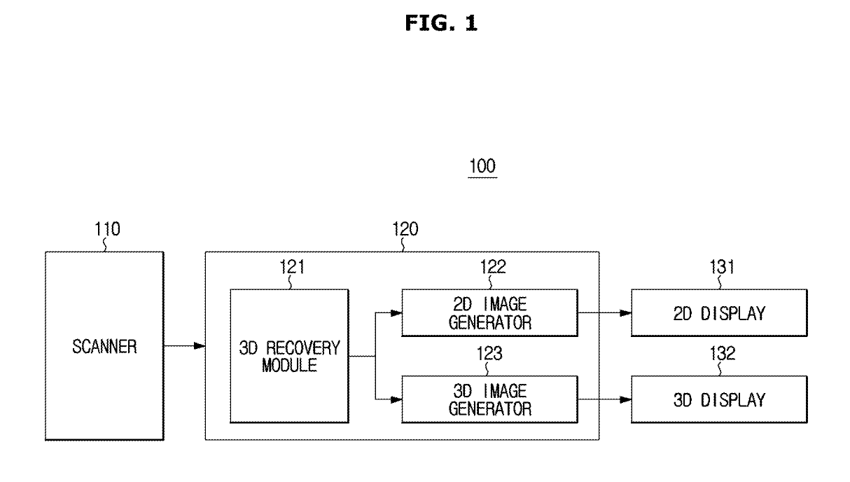

FIG. 1 is a control block diagram of a medical imaging apparatus according to an exemplary embodiment.

With reference to FIG. 1, a medical imaging apparatus 100 in accordance with an exemplary embodiment includes a scanner 110 acquiring projection data of the inside of an object by scanning the object, an image processor 120 recovering the volume of the object using the projection data and generating a two dimensional (2D) image and a three dimensional (3D) image of the object from the volume of the object, a 2D display 131 displaying the 2D image of the object, and a 3D display 132 displaying the 3D image of the object.

In accordance with an exemplary embodiment, the term “the object” refers to a region of a subject which is a target of diagnosis using the medical imaging apparatus 100. For example, when a region of a subject which is a target of diagnosis is the chest, the chest becomes the object, and when a region of a subject which is a target of diagnosis is the breast, the breast becomes the object. The subject may be a living body including, for example, a human body may become the subject of the medical imaging apparatus 100. An inner structure of the subject may be imaged by the medical imaging apparatus 100.

The image processor 120 includes a 3D recovery module 121 which three dimensionally recovers the volume of the object using the projection data of the object, a 2D image generator 122 which generates a 2D image of the object from the overall volume of the object, and a 3D image generator 123 which generates a 3D image of the object from the overall volume of the object. Here, the 2D image and the 3D image of the object are images of the inside of the object.

The image processor 120 may include one or more hardware and/or software components. For example, the image processor 120 may include one or more of an integrated circuitry, a dedicated circuit, firmware, and/or a processor such as a central processing unit (CPU) which executes software programs stored in a storage, e.g., a memory.

To image the inside of the object, projection data of the object may be first required. As described above, the scanner 110 acquires the projection data of the object by scanning the object. The scanner 110 may use radioactive rays or magnetic resonance to image the inside of the object.

To acquire sectional information and 3D structural information of the object, the scanner 110 scans the object from a plurality of different viewpoints.

For example, the scanner 110 may perform at least one from among computed tomography, positron emission tomography, and tomosynthesis using radioactive rays, and perform magnetic resonance imaging. Alternatively, at least two of the above imaging methods may be combined and performed. Hereinafter, a configuration and an operation of the scanner 110 according to exemplary embodiments will be described.

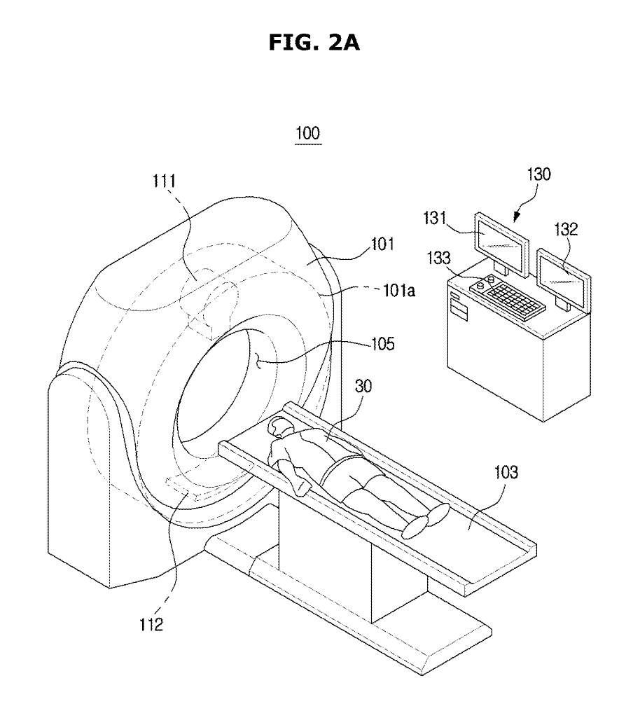

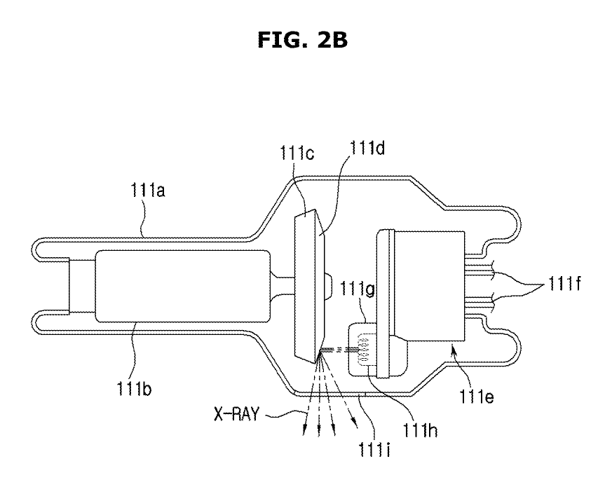

FIG. 2A is a perspective view illustrating an external appearance of a medical imaging apparatus including a scanner which performs computed tomography (CT) according to an exemplary embodiment, and FIG. 2B is a sectional view of a radiation source which radiates X-rays according to an exemplary embodiment.

When the scanner 110 performs computed tomography (CT), the scanner 110 includes a radiation source 111 which radiates radioactive rays to an object 30, and a radiation detector module 112 which detects radioactive rays transmitted through the object 30, as exemplarily shown in FIGS. 2A and 2B. The radiation source 111 and the radiation detector module 112 may be mounted on a gantry 101a such that radiation source 111 and the radiation detector module 112 face opposite to each other, and the gantry 101a is mounted within a housing 101.

When a table 103 on which the object 30 is located is fed to the inside of a bore 105 of the housing 101, the gantry 101a provided with the radiation source 111 and the radiation detector module 112 acquires projection data of the object 30 by scanning the object 30 while rotating at an angle of about 180 to about 360 degrees around the bore 105.

The radioactive rays include X-rays, γ-rays, α-rays, β-rays, and neutron beams. When the scanner 110 performs computed tomography (CT), the radiation source 111 may radiate X-rays.

When the radiation source 111 radiates X-rays, the X-ray source 111 may be a diode vacuum tube including an anode 111c and a cathode 111e, as exemplarily shown in FIG. 2B. The cathode 111e includes a filament 111h and a focusing electrode 111g which focuses electrons. The focusing electrode 111g may be referred to as a focusing cup.

A higher vacuum state having pressure of, for example, about 10 mmHg may be provided within a glass tube 111a, and the filament 111h of the cathode 111e may be heated to a higher temperature to generate thermal electrons. For example, a tungsten filament may be used as the filament 111h, and the filament 111h may be heated by applying current to an electric wire 111f connected to the filament 111h.

The anode 111c may mainly comprise copper, and a target material 111d may be applied to or disposed at a side of the anode 111c, which faces opposite to the cathode 111e. The target material 111d may comprise a higher resistance material, such as Cr, Fe, Co, Ni, W or Mo. When a melting point of the target material 111d is higher, a focal spot may have a decreased size. Here, the focal spot means an effective focal spot. Further, since the target material 111d is inclined at a designated angle, the size of the focal spot may be decreased when the inclination angle is smaller.

When higher voltage is applied to an area between the cathode 111e and the anode 111c, thermal electrons are accelerated and collide with the target material 111d of the anode 111c to generate X-rays. The generated X-rays are radiated to the outside through a window 111i. For example, the window 111i may comprise a beryllium (Be) thin film. Here, a filter (not shown) may be located on a front or rear surface of the window 111i to filter X-rays of a specific energy band.

The target material 111d may be rotated by a rotor 111b. When the target material 111d is rotated, a heat accumulation rate may be increased about 10 times or more per unit area, compared to when the target material is fixed.

Voltage applied to the area between the cathode 111e and the anode 111c of the X-ray source 111 is referred to as tube voltage, and an intensity of tube voltage may be expressed as kilovolt peak (kVp). When the tube voltage increases, a velocity of thermal electrons increases and consequently, energy of X-rays (or energy of photons) generated by collision of the thermal electrons with the target material 111d increases. Current flowing in the X-ray source 111 is referred to as tube current, and an intensity of tube current may be expressed as a mean value (mA) thereof. When the tube current increases, the number of thermal electrons discharged from the filament 111h increases and consequently, a dose of X-rays (or the number of X-ray photons) generated by collision of the thermal electrons with the target material 111d increases.

Therefore, since the energy of X-rays may be controlled by the tube voltage and the intensity or dose of X-rays may be controlled by the tube current and X-ray exposure time, the energy and intensity of radiated X-rays may be controlled according to kinds or characteristics of the object 30 or diagnosis purposes.

When the radiated X-rays have a designated energy band, the energy band may be defined by an upper limit and a lower limit. The upper limit of the energy band, i.e., a maximum energy of the radiated X-rays, may be adjusted by the intensity of the tube voltage, and the lower limit of the energy band, i.e., a minimum energy of the radiated X-rays, may be adjusted by the filter. When X-rays of a lower energy band are filtered by the filter, a mean value of the energy of the radiated X-rays may be increased.

The radiation detector module 112 may acquire projection data of the object 30 by detecting X-rays transmitted through the object 30, and transmit the acquired projection data to the image processor 120. Projection data acquired from a viewpoint represents a 2D projection image of the object 30. Since projection data from a plurality of viewpoints is acquired while the radiation source 111 and the radiation detector module 112 are rotated, the projection data transmitted to the image processor 120 represents a plurality of 2D projection images.

In computed tomography, the radiation detector module 112 may be referred to as a data acquisition system (DAS), and include a plurality of radiation detectors mounted on a frame in a one dimensional (1D) array. A detailed structure of the radiation detector will be described later.

When the scanner 110 performs positron emission tomography, a medicine containing a radioactive isotope which emits positrons is injected into a living body, and the radioactive isotope is traced by the scanner 110 such that distribution of the radioactive isotope within the body may be detected. In this case, an external appearance of the medical imaging apparatus 100 may be similar to that of the medical imaging apparatus 100 when the scanner 110 performs computed tomography (CT), shown in FIG. 2A.

The emitted positrons may disappear by coming electrons within the living body. When the positrons disappear, γ-rays are emitted in opposite directions. The emitted γ-rays pass through tissues in the living body, and the scanner 110 includes a radiation detector module detecting the γ-rays transmitted through tissues in the living body. Since a direction in which the γ-rays are emitted is not predicted, the radiation detector module in positron emission tomography may be arranged in a ring configuration in which a plurality of detectors may surround an object.

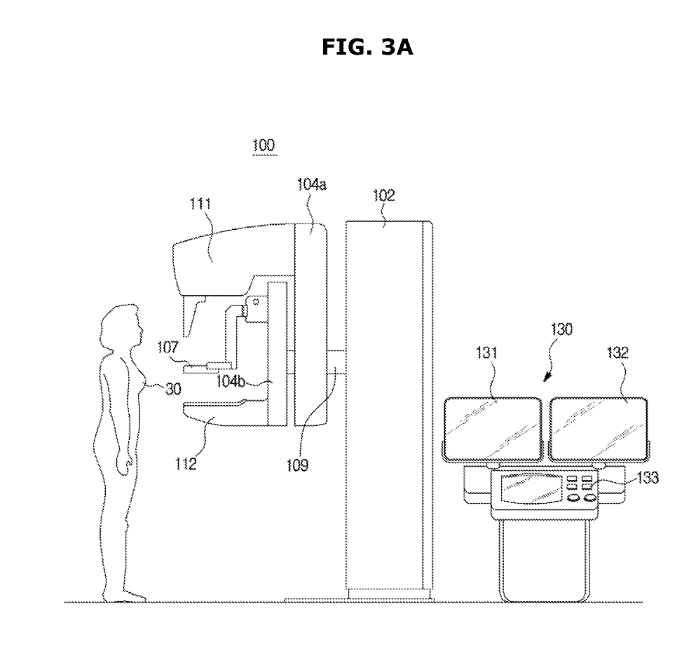

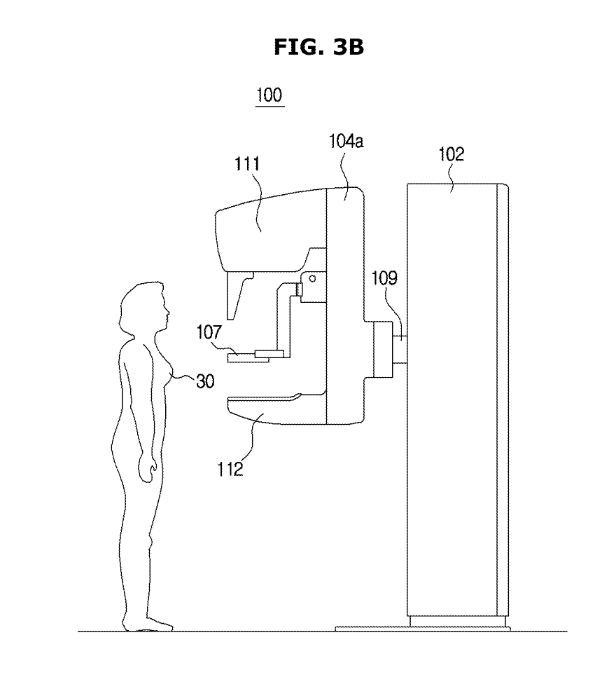

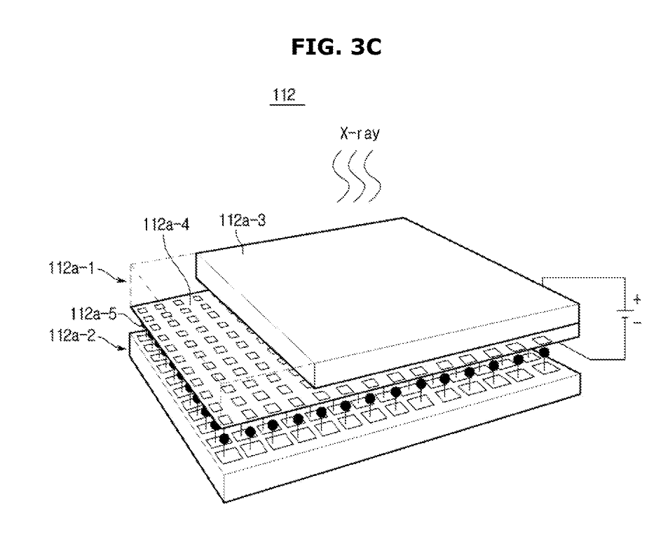

FIGS. 3A and 3B are views illustrating an external appearance of a medical imaging apparatus including a scanner which performs tomosynthesis according to an exemplary embodiment, and FIG. 3C is a view illustrating the structure of a radiation detector which detects X-rays according to an exemplary embodiment.

When the scanner 110 performs tomosynthesis, the scanner 110 may have a structure as shown in FIGS. 3A and 3B.

First, with reference to FIG. 3A, the scanner 110 includes a radiation source 111 which generates radioactive rays and radiates the radioactive rays to an object 30, and a radiation detector module 112 which detects radioactive rays transmitted through the object 30. The radiation source 111 may generate X-rays, and a configuration of the radiation source 111 may be substantially the same or similar to that of the radiation source 111 shown in FIG. 2B.

In a case of a breast comprising only soft tissues, to acquire clear images, the object 30, i.e., the breast, may be pressed using a pressing paddle 107. The pressing paddle 107 may move in an upward or downward direction along a second arm 104b connected to the radiation detector module 112. When the breast 30 is located on the radiation detector module 112, the pressing paddle 107 may move downward and press the breast 30 to have a designated thickness.

When the breast 30 is pressed, the radiation source 111 radiates X-rays, and the radiation detector module 112 detects X-rays transmitted through the breast 30. The radiation detector module 112 acquires projection data from the detected X-rays, and transmits the projection data to the image processor 120. The scanner 110 or the radiation source 111 is rotated at a designated angle, for example, about 20 to about 60 degrees, and the scanner 110 scans the object 30 from a plurality of different viewpoints during the rotation of the scanner 110 or the radiation source 111. Therefore, the projection data transmitted to the image processor 120 represent a plurality of 2D projection images of the object 30.

To scan the object 30 from a plurality of different viewpoints, a first arm 104a to which the radiation source 111 is connected may be rotated at a designated angle around a shaft 109 connected to a housing 102 and the rotation source 111 may radiate X-rays to the object 30 during the rotation of the first arm 104a. Here, the radiation detector module 112 may be fixed or rotated together with the first arm 104a. However, when the scanner 110 has a structure as shown in FIG. 3A, the radiation detector module 112 may be fixed and the radiation source 111 alone may be rotated.

Otherwise, in a case where in which both the radiation source 111 and the radiation detector module 112 are connected to the first arm 104a, as exemplarily shown in FIG. 3B, when the first arm 104 is rotated about the rotary shaft 109, both the radiation source 111 and the radiation detector module 112 are rotated.

The radiation detector module 112 may include radiation detectors which detect X-rays transmitted through the object 30, and a grid which substantially prevents scattering of X-rays.

Radiation detectors may be divided according to a material used for radiation detection, methods of converting detected X-rays into an electrical signal, and methods of acquiring an image signal.

For example, radiation detectors may be divided into a monolithic device type and a hybrid device type according to a material used for radiation detection.

In a case of a monolithic device type radiation detector, a part which detects X-rays and generates an electrical signal and a part which reads and processes the electrical signal may comprise the same semiconductor material or may be manufactured through the same process. For example, the monolithic device type radiation detector may use a light receiving device, such as a charge coupled device (CCD) or a complementary metal oxide semiconductor (CMOS).

In a case of a hybrid device type radiation detector, a part which detects X-rays and generates an electrical signal and a part which reads and processes the electrical signal may comprise different materials or may be manufactured through different processes. For example, the hybrid device type radiation detector may detect X-rays using a light receiving device, such as a photodiode or CdZnTe, and read and process an electrical signal using a CMOS read out integrated circuit (ROIC), detect X-rays using a strip detector, and read and process an electrical signal using at least one of from among the CMOS ROIC and an a-Si or a-Se flat panel system.

Further, radiation detectors are divided into a direct conversion type and an indirect conversion type according to methods of converting detected X-rays into an electrical signal.

In a direct conversion type radiation detector, when X-rays are radiated, electron-hole pairs are temporarily generated within a light receiving device, and electrons move to an anode and holes move to a cathode by an electric field applied to the light receiving device. The X-ray detector converts the movement of the electrons and the holes into an electrical signal. In the direct conversion type radiation detector, the light receiving device may use a-Se, CdZnTe, HgI₂, or PbI₂.

In an indirect conversion type radiation detector, a scintillator is provided between a light receiving device and the X-ray source, and when X-rays radiated from the X-ray source react with the scintillator and emit photons having a wavelength of a visible region, the light receiving device senses the photons and converts the photons into an electrical signal. In the indirect conversion type radiation detector, the light receiving device may use a-Si, and the scintillator may use a thin film type gadolinium oxysulphide (GADOX) scintillator, or micropillar-like or needle-like CSI(T1).

Further, radiation detectors may be divided according to operation modes such as, for example, a charge integration mode in which charges are stored for a designated time and a signal is acquired from the stored charges, and a photon counting mode in which photons having energy more than threshold energy are counted whenever a signal by an X-ray photon is generated, according to image signal acquisition methods.

The radiation detectors used in the medical imaging apparatus 100 in accordance with exemplary embodiments may include, but not limited to, any type of a radiation detector described above.

For example, referring to FIG. 3C, a radiation detector may include a light receiving device 112a-1 which detects radioactive rays and converts the detected radioactive rays into an electrical signal and a read circuit 112a-2 which reads the electrical signal. Here, the read circuit 112a-2 is provided in a 2D pixel array including a plurality of pixel areas. The light receiving device 112a-1 may comprise a single crystal semiconductor material to obtain a higher resolution, a faster response time and a higher dynamic range at a lower energy and a smaller dose. The single crystal semiconductor material used for the light receiving device 112a-1 may include, for example, Ge, CdTe, CdZnTe, or GaAs.

The light receiving device 112a-1 may comprise a PIN photodiode in which a p-type layer 112a-4, in which p-type semiconductors are arranged in a 2D pixel array, is bonded to a lower portion of a higher resistance n-type semiconductor substrate 112a-3, and the read circuit 112a-2 using a CMOS process may be combined with the light receiving device 112a-1 in each pixel. The CMOS read circuit 112a-2 and the light receiving device 112a-1 may be combined through a flip chip bonding method. That is, bumps 112a-5 comprising solder (PbSn) or indium (In) may be provided, and the CMOS read circuit 112a-2 and the light receiving device 112a-1 may be combined by reflowing the bumps 112a-5 and pressing the CMOS read circuit 112a-2 and the light receiving device 112a-1 under the condition that heat is applied to the bumps 112a-5. However, the above-described structure is only one example of the radiation detector 112a, and the radiation detector 112a according to exemplary embodiments is not limited thereto.

The above-described structure of the radiation detector 112a of FIG. 3C may be applied to the scanner 110 which performs computed tomography (CT).

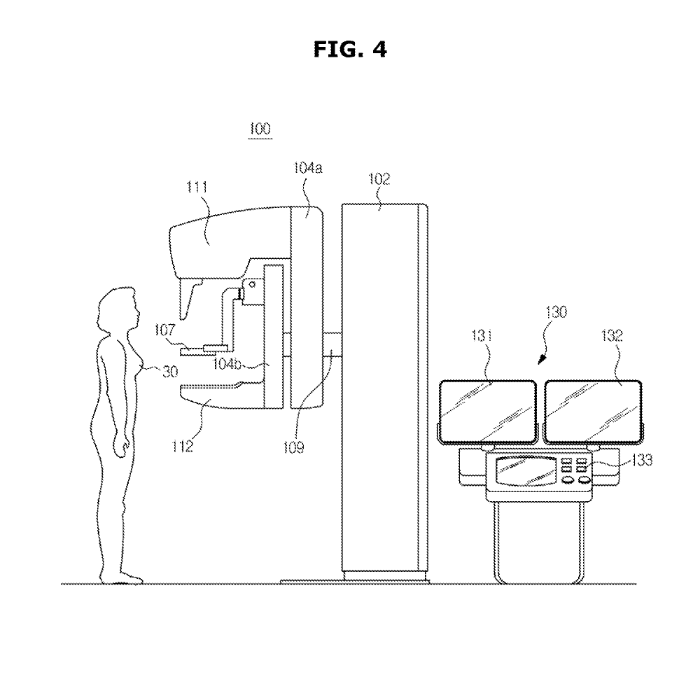

FIG. 4 is a view illustrating an external appearance of a medical imaging apparatus including a scanner which performs magnetic resonance imaging.

Referring to FIG. 4, in the scanner 110 which performs magnetic resonance imaging, the scanner 110 includes a magnet assembly mounted in the housing 101, and the magnet assembly 110 includes a static field coil 113 which provides a static magnetic field in the bore 105 of the housing 101, a gradient coil 113 which provides a gradient magnetic field by generating a gradient in the static magnetic field, and a radio frequency (RF) coil 115 which excites atomic nuclei by applying an RF pulse and receives an echo signal from the excited atomic nuclei. That is, when the table 103 on which the object 30 is located is fed to the inside of the bore 105, the static magnetic field, the gradient magnetic field, and the RF pulse may be applied to the object 30 such that atomic nuclei of the object 30 are excited, and an echo signal is generated from the excited atomic nuclei. The RF coil 115 receives the echo signal and transmits the received echo signal to the image processor 120. When the scanner 110 performs magnetic resonance imaging, the echo signal received by the RF coil 115 serves as projection data of the object 30.

Although not shown in FIG. 4, in the scanner 110 which performs magnetic resonance imaging, the medical imaging apparatus 100 may include a controller which controls an intensity and a direction of the static magnetic field, determines a pulse sequence, and controls the gradient coil 113 and the RF coil 115 according to the determined pulse sequence.

With reference to FIGS. 2A, 3A, 3B and 4, the medical imaging apparatus 100 includes a host device 130 which controls an overall operation of the scanner 110 or image processing. The host device 130 may include the 2D display 131, the 3D display 132, and an input unit 133 which receives control instructions input by a user.

In the above, the configuration and the operation of the scanner 110 which acquires projection data of an object by scanning the object according to exemplary embodiments have been described in detail. Hereinafter, a configuration and an operation of the image processor 120 which images the inside of the object will be described in detail.

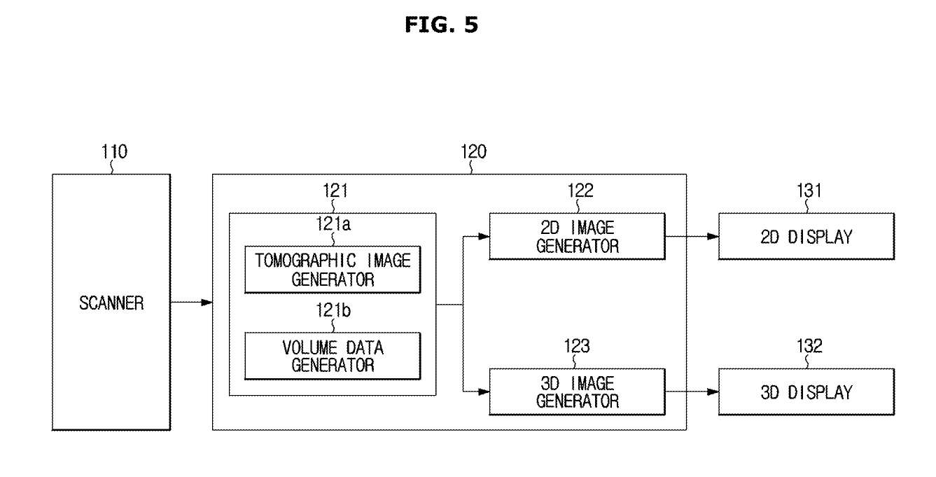

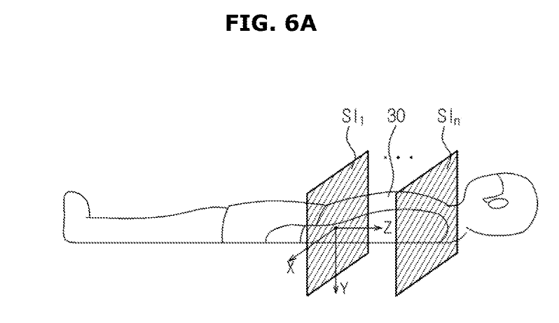

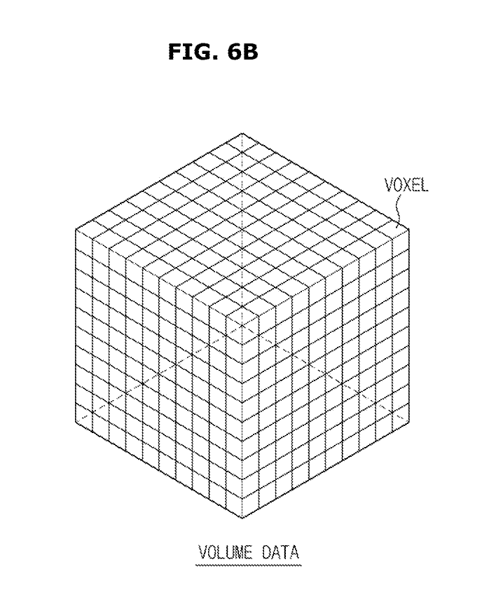

FIG. 5 is a control block diagram illustrating a 3D recovery module according to an exemplary embodiment in detail, FIG. 6A is a schematic view illustrating tomographic images of an object according to an exemplary embodiment, and FIG. 6B is a schematic view illustrating a recovered volume of an object according to an exemplary embodiment.

The projection data acquired by the scanner 110 by scanning the object is transmitted to the 3D recovery module 121. As exemplarily shown in FIG. 5, the 3D recovery module 121 may include a tomographic image generator 121a which generates tomographic images of the object and a volume data generator 121b which generates volume data of the object from the tomographic images of the object.

As described above, the scanner 110 acquires projection data from a plurality of different viewpoints using the structure of the scanner 110 configured to rotate about the object 30 at a designated angle or configured to surround the object 30. The tomographic image generator 121a may generate tomographic images of the object 30 by reconstructing the projection data transmitted from the scanner 110. The tomographic image is an image which represents a section of the object. Hereinafter, for convenience of description, an image generated by reconstructing projection data will be referred to as a tomographic image.

Methods of reconstructing projection data in the tomographic image generator 121a may include, for example, an iterative method, a direct Fourier method, a back projection method, and a filtered back projection method.

In the iterative method, projection data may be continuously compensated for until data closer to an original structure of an object is acquired. In the back projection method, pieces of projection data acquired from a plurality of viewpoints may be gathered on a screen. In the direct Fourier method, projection data may be converted from a space domain to a frequency domain. In the filtered back projection method, to offset blur around a center of the projection data, back projection may be performed after a filtering operation is performed on the projection data.

Since scanning is carried out in a region of the object having a designated area, the tomographic image generator 121a may generate a plurality of tomographic images corresponding to different positions.

For example, with reference to FIG. 6A, when the object is a chest of a human body and the human body is fed into the bore 105 to be scanned, projection data of a region having a designated area on an X-Z plane may be acquired, and thus, a plurality of tomographic images SI₁ to SIₙ on the X-Y plane in a Z direction may be generated.

The volume data generator 121b three dimensionally recovers a volume of the object using the plurality of tomographic images. When the plurality of tomographic images are cross-sectional images of the object, the volume of the object may be three dimensionally recovered by accumulating a plurality of sectional images in a longitudinal direction thereof. In an exemplary embodiment shown in FIG. 6A, the volume of the object may be recovered by accumulating the plurality of sectional images SI₁ to SIₙ in the Z direction.

With reference to FIG. 6B, the volume of the object may be expressed by volume data, and the volume data comprises a group of pieces of data which are three dimensionally arranged. Data included in the volume data is referred to as a voxel, and the voxel has a scalar value or a vector value sampled by a designated interval.

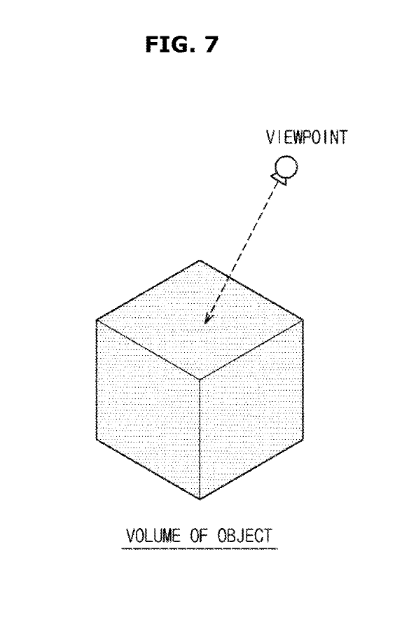

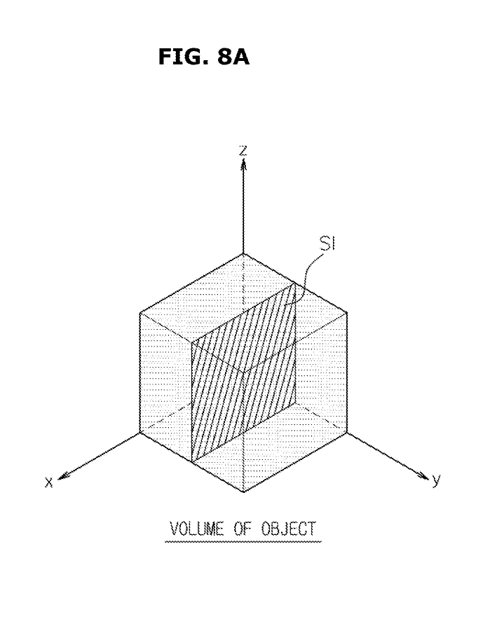

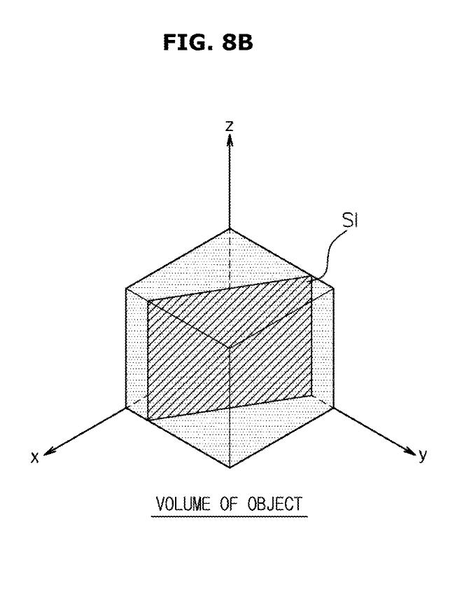

Hereinafter, an operation of the 2D image generator 122 will be described in detail with reference to FIGS. 7, 8A, and 8B.

FIG. 7 is a schematic view illustrating a volume of an object rendered from a viewpoint according to an exemplary embodiment.

As an example, as exemplarily shown in FIG. 7, the 2D image generator 122 may perform rendering of the volume of the object from a viewpoint to generate a 2D image. Thus, the rendered volume of the object has an effect such that the volume of the object is shown as being seen from the corresponding viewpoint. The viewpoint from which volume rendering is performed may be a virtual viewpoint. Since volume rendering from a viewpoint may be regarded as projection of the volume of the object from the corresponding viewpoint, a 2D image generated by volume rendering will be referred to as a reprojection image hereinafter.

The volume rendering is an operation of visualizing 3D volume data as a 2D image, and is generally divided into surface rendering and direct rendering. In the surface rendering, surface information may be estimated from volume data based on a scalar value set by a user and a spatial variation. The surface information is changed to a geometric factor, such as a polygon or a curved patch, and thus visualized. A representative surface rendering method includes, for example, a marching cube algorithm.

In the direct rendering, volume data may be directly visualized without converting surface information to a geometric factor. The direct rendering may be divided into an image-order algorithm and an object-order algorithm according to volume data searching methods.

In the object-order algorithm, volume data may be searched in an order of storage, and thus respective voxels may be combined with corresponding pixels. A representative object-order algorithm may include, for example, splatting.

In the image-order algorithm, respective pixel values may be determined in an order of scan lines of an image. That is, the pixel values corresponding to volume data may be sequentially determined based on virtual rays from respective pixels. A representative image-order algorithm may include, for example, ray casting and ray tracing. When the ray casting and the ray tracing are performed, rays of various wavelength bands, for example, visible rays or X-rays, may be applied.

The ray casting is a method in which virtual rays from respective pixels of an image plane are radiated, colors and opacities at respective sampling points on the rays are calculated, and values of corresponding pixels are determined by combining the calculated colors and opacities. Here, radiation methods, i.e., projection methods, of rays may include, for example, a parallel projection method and a perspective projection method.

The ray tracing is a method in which paths of rays which reach eyes of a viewer are respectively traced. That is, differently from the ray casting in which intersection points between rays and the volume of an object are detected, the ray tracing may use reflection or refraction of rays by respectively tracing paths of radiated rays.

The ray tracing may be divided into forward ray tracing and backward ray tracing. The forward ray tracing is a technique in which rays which reach eyes of a viewer are detected by modeling various optical phenomena such as, for example, reflection, scattering, transmission of rays radiated from a virtual light source to an object, and the backward ray tracing is a technique in which paths of rays which reach eyes of a viewer are traced in a backward direction.

The 2D image generator 122 may generate a reprojection image from a viewpoint by applying any of the above-described volume rendering methods.

The viewpoint from which volume rendering is performed may be set through the input unit 133 by a user, or may be arbitrarily set by the 2D image generator 122. The input unit 303 may include, for example, a keyboard, a mouse, a trackball, a touch screen, a microphone, and the like. A user who diagnoses a subject using the medical imaging apparatus 100 in accordance with an exemplary embodiment may be one of medical personnel including a doctor, a radiologist, and a nurse, but is not limited thereto. That is, the user may be any person who uses the medical imaging apparatus 100.

FIGS. 8A and 8B are schematic views illustrating a sectional image generated from a volume of an object.

As an example, as exemplarily shown in FIGS. 8A and 8B, the 2D image generator 122 may generate a sectional image SI corresponding to an arbitrary plane from the volume of an object. In this case, a 2D image generated by the 2D image generator 122 may correspond to a sectional image of the object.

With reference to FIG. 8A, the 2D image generator 122 may generate the sectional image SI corresponding to an X-Z plane from the volume of an object located in a 3D space expressed by X, Y and Z axes. Alternatively, the 2D image generator 122 may generate the sectional image SI corresponding to an X-Y plane or a Y-Z plane. Further alternatively, with reference to FIG. 8B, the 2D image generator 122 may generate a sectional image SI corresponding to an arbitrary plane other than the X-Y plane, the X-Z plane, or the Y-Z plane.

A region represented by the sectional image generated by the 2D image generator 122 may be set by a user through the input unit 133. Therefore, a user may acquire a sectional image required in diagnosis by setting a region for generating the sectional image.

The 2D image generated by the 2D image generator 122 may be displayed through the 2D display 131. The 2D display 131 may be a display device including a display such as, for example, a liquid crystal display (LCD), a light emitting diode (LED), an organic light emitting diode (OLED), a plasma display panel (PDP), or a cathode ray tube (CRT).

The 2D display 131 may display tomographic images of the object generated by the tomographic image generator 121a.

A user may identify information or an overall structure of a region in detail through sectional images, reprojection images, or tomographic images, displayed on the 2D display 131.

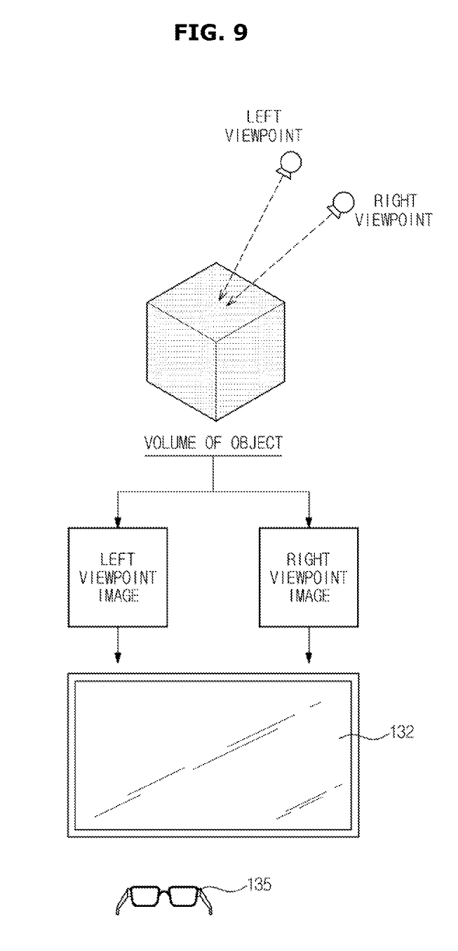

Hereinafter, with reference to FIGS. 9 to 13, generation of a 3D image of an object by the 3D image generator 123 will be described in detail.

The 3D image generator 123 generates a plurality of reprojection images corresponding to a plurality of viewpoints by performing rendering of the volume of an object from the corresponding plurality of viewpoints. A 3D image generated by the 3D image generator 123 is an image which may be seen as a 3D image when the image is output through the 3D display 132, and the plurality of reprojection images correspond to the 3D image.

Volume rendering performed by the 3D image generator 123 may be a method that is substantially the same or different from volume rendering performed by the 2D image generator 122. For example, a plurality of reprojection images may be generated by radiating virtual X-rays to the volume of the object using ray casting or ray tracing.

FIG. 9 is a schematic view illustrating a process of rendering a volume from a right viewpoint and a left viewpoint according to an exemplary embodiment.

The number of a plurality of viewpoints from which volume rendering is performed may be determined by an output format of the 3D display 132. For example, when the 3D display 132 has an output format corresponding to a stereoscopic method, the 3D image generator 123 may perform rendering of the volume of an object from a right viewpoint and a left viewpoint corresponding to a right eye and a left eye of a viewer, and thus generate a reprojection image corresponding to the right viewpoint, i.e., a right viewpoint image, and a reprojection image corresponding to the left viewpoint, i.e., a left viewpoint image. The generated right viewpoint image and left viewpoint image are input to the 3D display 132.

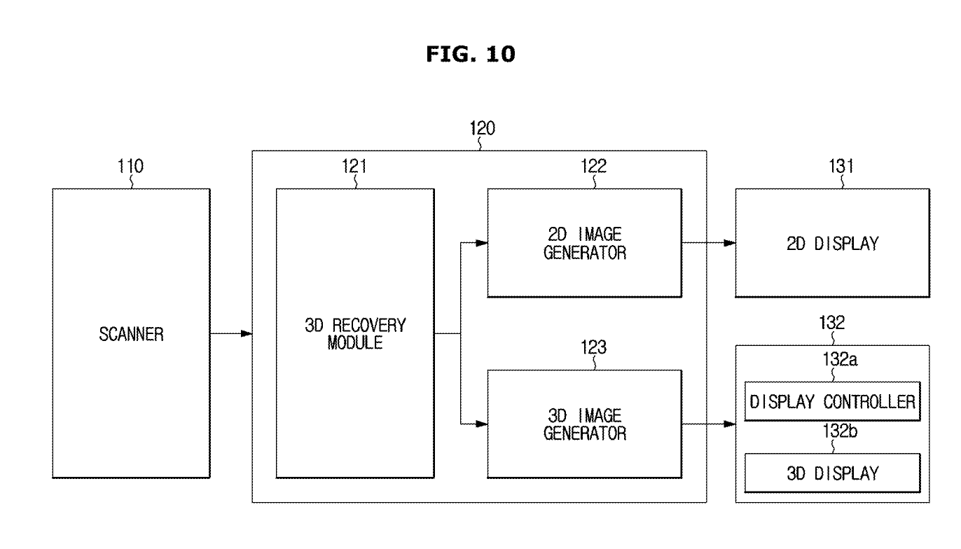

FIG. 10 is a control block diagram illustrating a configuration of a 3D display according to an exemplary embodiment.

With reference to FIG. 10, the 3D display 132 includes a 3D display 132b which three dimensionally displays the right viewpoint image and the left viewpoint image, and a display controller 132a controls the 3D display 132b.

With reference to FIG. 9, when the 3D display 132 employs the stereoscopic method, a viewer may three dimensionally view images displayed through the 3D display 132 by wearing 3D glasses 135.

In more detail, the stereoscopic method may be divided into a polarized glass method and a shutter glass method. In a case of the polarized glass method, the display controller 132a may divide scanning lines of the 3D display 132b into even numbered lines and odd numbered lines, and displays the left viewpoint image and the right viewpoint image on the respective even numbered lines and the odd numbered lines. Thus, the left viewpoint image and the right viewpoint image may be simultaneously displayed through the 3D display 132b. A polarizing filter which separately outputs the two images is attached to a front surface of the display 132b, and different polarizing plates are respectively mounted on left and right lenses of the 3D glasses 135. For example, the left viewpoint image may be displayed through only the left lens, and the right viewpoint image may be displayed through only the right lens.

When the shutter glass method is applied, the display controller 132a alternately displays the left viewpoint image and the right viewpoint image through the 3D display 132b. Here, a shutter mounted on the 3D glasses 135 is synchronized with the 3D display 132, and is selectively opened and closed according to the left viewpoint image or the right viewpoint image displayed through the 3D display 132b.

Further, the 3D display 132 may employ an autostereoscopic method without 3D glasses. The autostereoscopic method may be divided into a multi-view method, a volumetric method, and an integral image method.

When the 3D display 132 employs the multi-view method, the 3D image generator 123 generates a multi-view 3D image and outputs the generated multi-view 3D image to the 3D display 132.

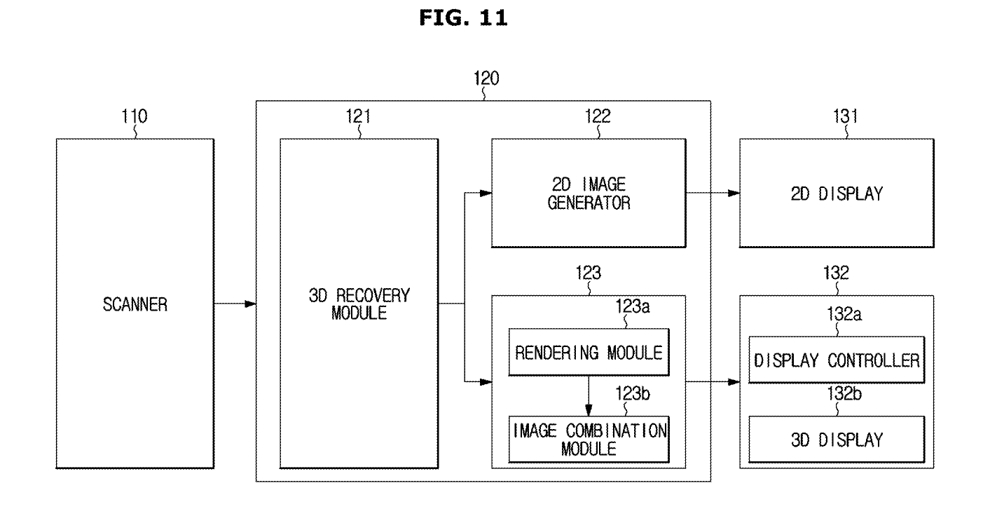

FIG. 11 is a control block diagram illustrating a configuration of a 3D image generator when the multi-view method is employed according to an exemplary embodiment.

With reference to FIG. 11, the 3D image generator 123 of the medical imaging apparatus 100 according to an exemplary embodiment may include a rendering module 123a which performs rendering of the volume of an object from a plurality of viewpoints and an image combination module 123b which generates a multi-view 3D image by combining a plurality of reprojection images generated by the volume rendering.

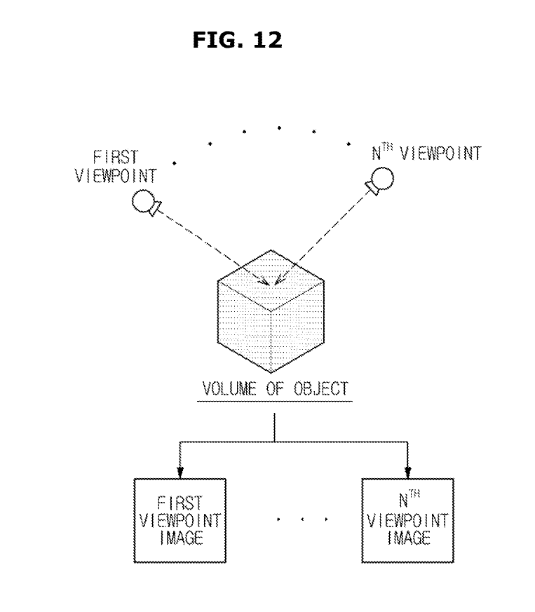

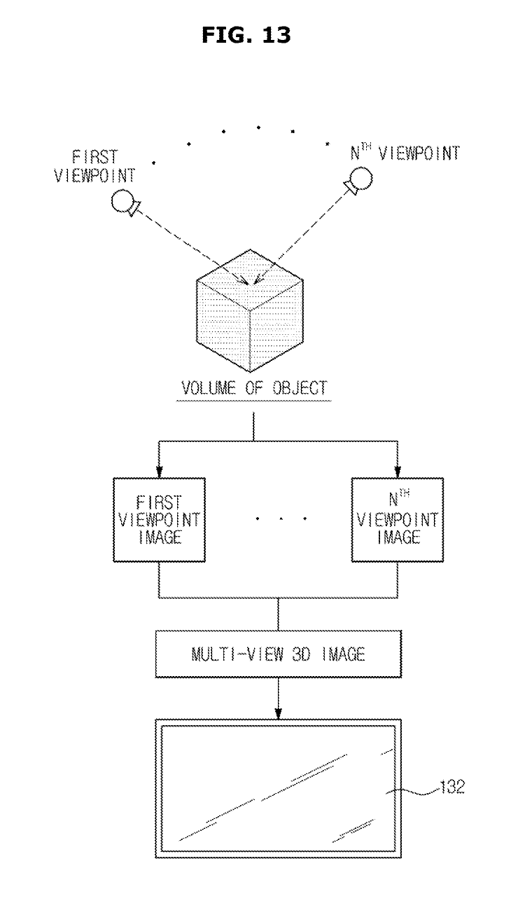

FIG. 12 is a schematic view illustrating a process of generating a plurality of reprojection images by rendering a volume of an object according to an exemplary embodiment, and FIG. 13 is a schematic view illustrating a process of combining a plurality of reprojection images to generate a multi-view 3D image according to an exemplary embodiment.

With reference to FIG. 12, the rendering module 123a according to an exemplary embodiment may perform rendering of the volume of the object from n viewpoints (n being an integral number more than 2), and thus generate n reprojection images corresponding to the respective n viewpoints. Specifically, the rendering module 123a may generate a reprojection image corresponding to a first viewpoint (i.e., a first viewpoint image) to a reprojection image corresponding to an nth viewpoint (i.e., an nth viewpoint image).

With reference to FIG. 13, the image combination module 123b according to an exemplary embodiment generates a multi-view 3D image by combining the first viewpoint image to the nth viewpoint image. To combine images corresponding to respective viewpoints, a technique of weaving the images may be used. The multi-view 3D image is output to the 3D display 132, and the 3D display 132 three dimensionally displays the multi-view 3D image.

The 3D display 132 may include a lenticular lens or a parallax barrier installed on the front surface of the 3D display 132b. The lenticular lens is used to separate left and right images from each other by gathering light, and the parallax barrier is used to separate left and right images from each other by blocking light, thus allowing a viewer to feel a 3-dimensional effect without 3D glasses.

A user may detect detailed information of a region of interest from a sectional image or a projection image from a viewpoint, displayed through the 2D display 131, and may detect an overall contour and depth information of the object from the 3D image displayed through the 3D display 132. That is, a user may obtain information required for accurate diagnosis at a glance, and thus accuracy and efficiency in diagnosis may be improved.

Hereinafter, a control method of a medical imaging apparatus in accordance with the embodiment will be described.

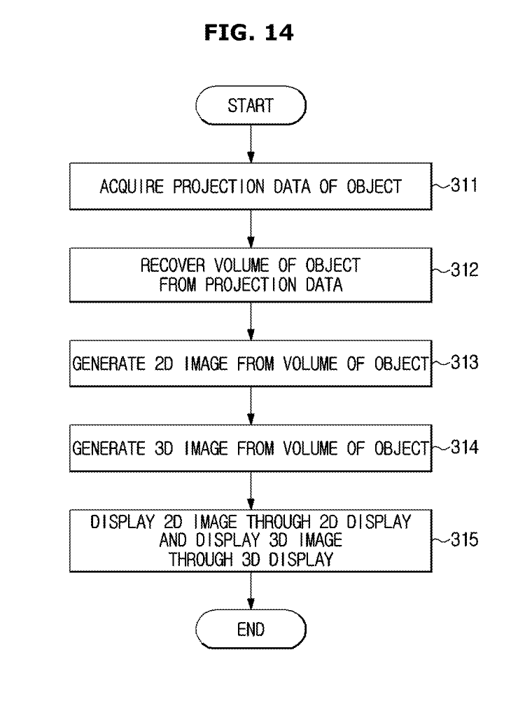

FIG. 14 is a flowchart illustrating a control method of a medical imaging apparatus according to an exemplary embodiment.

With reference to FIG. 14, projection data of an object is first acquired (operation 311). The projection data may be acquired by scanning the object from a plurality of different viewpoints, and scanning of the object may be performed by at least one from among, for example, computed tomography, positron emission tomography, and tomosynthesis using radioactive rays, or magnetic resonance imaging.

The volume of the object may be recovered using the projection data (operation 312). To recover the volume of the object, a plurality of tomographic images may be generated by reconstructing the projection data, and volume data may be generated by accumulating the plurality of tomographic images. The volume of the object may include volume data which is three dimensionally arranged. Reconstruction of the projection data and generation of the volume data have been described above in the medical imaging apparatus 100 in accordance with an exemplary embodiments and thus will be omitted.

A 2D image is generated from the volume of the object (operation 313). The 2D image may be a reprojection image generated by performing rendering of the volume of the object from at least one viewpoint, or a sectional image corresponding to at least one plane of the volume of the object. The volume rendering has been described above in the medical imaging apparatus 100 in accordance with an exemplary embodiment and thus will be omitted now.

A 3D image may be generated from the volume of the object (operation 314). The 3D image may be a plurality of reprojection images generated by performing rendering of the volume of the object from a plurality of viewpoints or a multi-view 3D image generated by combining the plurality of reprojection images. Although FIG. 14 illustrates that the 3D image is performed after the 2D image is generated, exemplary embodiments are not limited thereto. For example, in alternative embodiments, the 2D image and the 3D image may be simultaneously generated, or the 2D image may be generated prior to generation of the 3D image. That is, in various exemplary embodiments, a generation sequence of the 2D image and the 3D image may be varied.

The 2D image may be displayed through the 2D display 131, and the 3D image may be displayed through the 3D display 132 (operation 315). A user may detect detailed information of a region of interest from the sectional image or the projection image from a viewpoint, displayed through the 2D display 131, and may detect an overall contour and depth information of the object from the 3D image displayed through the 3D display 132. That is, a user may obtain information required for accurate diagnosis at a glance, and thus accuracy and efficiency in diagnosis may be improved.

According to exemplary embodiments, a medical imaging apparatus may include a 2D display device which displays a 2D image of an object and a 3D display device which displays a 3D image of the object. Thus, the 2D image and the 3D image may be identified during diagnosis, thereby promoting accuracy and promptness of diagnosis.

Exemplary embodiments may also be implemented through computer-readable recording media having recorded thereon computer-executable instructions such as program modules that are executed by a computer. Computer-readable media may be any available media that can be accessed by a computer and include both volatile and nonvolatile media and both detachable and non-detachable media. Examples of the computer-readable media may include a read-only memory (ROM), a random-access memory (RAM), a compact disc (CD)-ROM, a magnetic tape, a floppy disk, an optical data storage device, etc. Furthermore, the computer-readable media may include computer storage media and communication media. The computer storage media include both volatile and nonvolatile and both detachable and non-detachable media implemented by any method or technique for storing information such as computer-readable instructions, data structures, program modules or other data. The communication media typically embody computer-readable instructions, data structures, program modules, other data of a modulated data signal such as a carrier wave, or other transmission mechanism, and they include any information transmission media.

Although a few exemplary embodiments have been shown and described, it would be appreciated by those skilled in the art that changes may be made in these embodiments without departing from the principles and spirit of the disclosure, the scope of which is defined in the claims and their equivalents.

What is claimed is:

1. A medical imaging apparatus comprising:
a scanner configured to acquire a projection data of an object;
a three dimensional (3D) recovery module configured to recover a volume of the object based on the projection data;
a two dimensional (2D) image generator configured to generate a 2D image of the object based on the volume of the object;
a 3D image generator configured to generate a 3D image of the object based on the volume of the object;
a 2D display configured to display the 2D image of the object; and
a 3D display configured to display the 3D image of the object.

2. The medical imaging apparatus according to claim 1, wherein the scanner acquires a plurality of projection data from a plurality of different viewpoints.

3. The medical imaging apparatus according to claim 2, wherein the 3D recovery module includes:
a tomographic image generator configured to generate a plurality of tomographic images of the object by reconstructing the plurality of projection data; and
a volume data generator configured to generate a volume data corresponding to the volume of the object based on the plurality of tomographic images.

4. The medical imaging apparatus according to claim 1, wherein the 2D image generator generates at least one reprojection image corresponding to at least one viewpoint by rendering the volume of the object from the at least one viewpoint.

5. The medical imaging apparatus according to claim 1, wherein the 2D image generator generates at least one sectional image corresponding to at least one plane from the volume of the object.

6. The medical imaging apparatus according to claim 4, wherein the 3D image generator generates a plurality of reprojection images corresponding to a plurality of different viewpoints by rendering the volume of the object from the plurality of viewpoints.

7. The medical imaging apparatus according to claim 6, wherein the 3D image generator generates a multi-view 3D image based on the plurality of reprojection images.

8. The medical imaging apparatus according to claim 6, wherein the plurality of reprojection images include a first reprojection image corresponding to a left viewpoint and a second reprojection image corresponding to a right viewpoint.

9. The medical imaging apparatus according to claim 8, wherein the 3D display substantially simultaneously displays the first reprojection image corresponding to the left viewpoint and the second reprojection image corresponding to the right viewpoint

10. The medical imaging apparatus according to claim 8, wherein the 3D display alternately displays the first reprojection image corresponding to the left viewpoint and the second reprojection image corresponding to the right viewpoint.

11. The medical imaging apparatus according to claim 7, wherein the 3D display displays the multi-view 3D image, and
a lenticular lens or a parallax barrier is provided on a front surface of the 3D display.

12. The medical imaging apparatus according to claim 1, wherein the scanner acquires the projection data of the object by performing at least one from among computed tomography, positron emission tomography, tomosynthesis, and magnetic resonance imaging.

13. A control method of a medical imaging apparatus, the control method comprising:
acquiring projection data of an object;
recovering a volume of the object based on the projection data;
generating a 2D image of the object based on the volume of the object;
generating a 3D image of the object based on the volume of the object; and
displaying the 2D image of the object through a 2D display, and displaying the 3D image of the object through a 3D display.

14. The control method according to claim 13, wherein the generating the 2D image of the object comprises generating at least one reprojection image corresponding to at least one viewpoint by rendering the volume of the object from the at least one viewpoint.

15. The control method according to claim 13, wherein the generating the 2D image of the object comprises generating at least one sectional image corresponding to at least one plane from the volume of the object.

16. The control method according to claim 14, wherein the generating the 3D image of the object comprises generating a plurality of reprojection images corresponding to a plurality of different viewpoints by rendering the volume of the object from the plurality of viewpoints.

17. The control method according to claim 16, wherein the generating the 3D image of the object further comprises generating a multi-view 3D image based on the plurality of reprojection images.

18. An image processing apparatus comprising:
at least one processor which implements:
a three dimensional (3D) recovery module configured to obtain a volume of an object based on a plurality of tomographic images of the object scanned from a plurality of viewpoints;
an image generator configured to generate at least one from among a two dimensional (2D) image and a three dimensional (3D) image of the object based on the volume of the object.

19. The image processing apparatus according to claim 18, wherein the image generator comprises a 2D image generator configured to generate the 2D image of the object based on the volume of the object, and
wherein the 2D image generator generates the 2D image of the object based on at least one from among a tomographic image of the object, an image obtained by rendering the volume of the object from a viewpoint, and an image corresponding to a sectional plane of the volume of the object.

20. The image processing apparatus according to claim 18, wherein the mage generator comprises a 3D image generator configured to generate the 3D image of the object based on the volume of the object, and
the 3D image generator generates the 3D image of the object based on a plurality of images obtained by rendering the volume of the object from a second plurality of viewpoints.
